(19) United States
(12) Patent Application Publication
(10) Pub. No.: US 2009/0301481 A1
(43) Pub. Date: Dec. 10, 2009

(54) Medicant dispenser, and a device and method for monitoring dispensing of a medicant from a dispenser

(76) Inventor: Michael Hennessy Sullivan, Worcestershire (GB)

Correspondence Address: HOFFMAN WARNICK LLC, 75 STATE STREET, 14TH FLOOR, ALBANY, NY, 12207, US

(21) Appl. No.: 12/066,617

(22) PCT Filed: Sep. 12, 2006
(86) PCT No.: PCT/GB06/03368

§ 371 Date: Sep. 4, 2008

(30) Foreign Application Priority Data
Sep. 12, 2005 (GB) 0518566.5

(51) Int. Cl. A61M 15/00

(52) U.S. Cl. 128/203.12

(57) ABSTRACT

A medicant dispenser comprises a valve with an actuator movable between a valve closed and a valve open position and through which medicant is selectively released and a device for detecting that medicant has been released. The device comprises a light generator for emitting a beam of light, a sensor for sensing light emitted from the light generator and a counter coupled to the sensor for receiving a signal therefrom and for counting dispensation of the medicant. The sensor is arranged to switch the counter to an operating condition on receipt of light emitted from the light generator. The light generator and the sensor are positioned such that medicant being dispensed interrupts the light beam emitted from the light generator thereby provoking a further signal from the sensor. The counter is arranged to detect variations in the further signal from the sensor indicative of dispensation of the medicant. There is also disclosed a method of monitoring dispensation of a medicant from a medicant dispenser and a device for detecting that medicant has been released from a medicant dispenser.

### CLAIM FOR PRIORITY

This application claims priority to International Application PCT/GB2006/003368, with an International Filing Date of Sep. 12, 2006, which claims priority to United Kingdom patent application number 0518566.5, filed on Sep. 12, 2005, both of which are hereby incorporated by reference herein.

The present invention relates to a medicant dispenser, a device for monitoring dispensing of a medicant from a dispenser, and a method of monitoring dispensation of a medicant from a medicant dispenser.

In inhalers and other medical devices where it is important that the dosage delivery of a medicant is precisely controlled, the operation of any valve through which the medicant is delivered should be carefully monitored as well as the delivery of the medicant. In particular, in devices which include a dosage counter, it is important that actual doses be counted and not just the valve operations.

International patent application WO 03/090824 discloses a system for dispensing medicaments having a housing with an outlet, a medicament container locatable within the housing, and an electronic dose counter associated with the outlet. The dose counter includes a first sensor for directly detecting the medicament released from the container through the outlet and a measurement device for detecting the changes in performance of the sensor attributable to contamination or degradation of the sensor and for adjusting its operation to compensate for such changes. An optical beam is used to count the doses by detecting the interruption of the beam by the medicament as it is dispensed through the outlet. The described system has complex electronics for determining and correcting the variations in received signal strength due to contamination and degradation of the sensor and is therefore costly.

International patent application WO01/93932 is directed to an aerosol drug delivery system comprising a frame holding a disposable container containing the drug formulation, an outlet, control electronics including a dosage counter, and an optical detection system to detect movement of the container. When the medicament is dispensed, it passes through a light beam emitted from an emitter, thereby interrupting the light beam. This interruption is detected by a detector which triggers the dosage counter. In this system, the control electronics and counting electronics are normally maintained in the sleep mode and are awakened by pressing a status button. This system requires a manual awakening of the electronics before being in an operable condition and has complex electronics which increases the cost of production.

The present applicant's co-pending International patent application number PCT/GB2005/000790 is directed to a system for dispensing a medicant. The described system includes a pressurised canister containing the medicant to be dispensed, a valve attached to the canister through which the medicant is dispensed, and an LED mounted on the valve together with a piezo crystal or photovoltaic polymer which generates the voltage to operate the LED on activation of the valve. The container and the valve are mounted in a housing which also contains a detector and a dosage counter. When the valve is fully opened, light emitted from the LED passes through an aperture in the valve mechanism and through a corresponding aperture in the housing and on the detector indicating that the valve is fully open. The output from the detector operates the dosage counter. Although the described system confirms that the valve is fully open, as otherwise the two apertures would not align, it does not detect whether or not any medicant has been dispensed from the valve and thereby is predominantly concerned with monitoring valve operation.

The present applicant's co-pending International patent application number PCT/GB2005/000793 is also directed to a system for dispensing a medicant, and presents an alternative way of monitoring valve operation to that described in PCT/GB2005/000790. The system includes a pressurised canister containing the medicant to be dispensed, a valve mechanism attached to the canister through which medicant may be dispensed and a housing for supporting the canister. A light source is mounted on the valve mechanism or the pressurised canister and, in one embodiment, the motion of the beam emitted from the light source is determined by a detector or array of detectors mounted on the housing. In a further described embodiment, light from an LED mounted on the housing may be reflected by a mirror onto a detector or an array of detectors mounted on the housing. Although the described system confirms that the valve is fully open, it does not detect whether or not any medicant has been dispensed from the valve and is predominantly concerned with monitoring valve operation.

With systems such as those described in International patent application number PCT/GB2005/000793, as individual valves will not all have precisely the same open and closed positions, it is necessary to individually calibrate the systems to ensure accurate monitoring of the valve usage. In this system, the calibration is accomplished in the combination of the valve and monitoring device when the valve is first operated. This may be time-consuming.

Thus there is a need for a system which is less complex to manufacture and use whilst at the same time being able to detect dispensing of a dose of medicant rather than merely the opening of the valve.

In general terms, the present invention resides in a device, a medicant dispenser and a method of dispensing a medicant in which a light generator and a sensor are positioned such that medicant being dispensed interrupts the light beam emitted from the light generator thereby provoking a further signal from the sensor; a counter being arranged to detect variations in the further signal from the sensor indicative of dispensation of the medicant.

This is particularly advantageous over the prior art systems described above as it provides a less complex system, device and method of monitoring dosage dispensation, requiring fewer electronics and no need for an additional switching component to activate the counter. Thus preferred embodiments of the present invention result in a device which is more compact, more economical to manufacture and less complex to use than the conventional systems and methods described above.

According to a first aspect of the present invention there is provided a medicant dispenser comprising:
a valve with an actuator movable between a valve closed and a valve open position and through which medicant is selectively released; and
a device for detecting that medicant has been released; wherein the device comprises:
   a light generator for emitting a beam of light;
   a sensor for sensing light emitted from the light generator; and
   a counter coupled to the sensor for receiving a signal therefrom and for counting dispensation of the medicant;
   the light generator, the sensor and the counter being arranged to be switchable between a rest condition and an operating condition; the light generator and the sensor being positioned such that medicant being dispensed interrupts the light beam emitted from the light generator thereby provoking a further signal from the sensor; the counter being arranged to detect variations in the further signal from the sensor indicative of dispensation of the medicant.

Preferably, the medicant dispenser further comprises a switch for activating power to the light generator.

The light generator may comprise, for example, a light emitting diode (LED).

Preferably, the sensor is arranged to switch the counter to the operating condition on receipt of light emitted from the light generator.

Alternatively, in a further preferred embodiment, the counter is arranged to switch the light generator to the operating condition.

Preferably, the medicant dispenser further comprises a battery arranged to power the light generator.

In a preferred embodiment, the device and the valve are retained in a housing and the sensor and/or the light generator may be mountable on opposing faces of the housing.

Alternatively, the light generator may be mountable on a section attachable to a canister containing the medicant to be dispensed which is insertable in use in the housing.

In a preferred embodiment, the sensor is mountable on a face of the housing opposing the light generator and a filter is mountable adjacent the sensor, the filter being positionable such that light from the light generator passes through the filter before being received by the sensor.

In a further preferred embodiment, the medicant dispenser comprises a first light generator and a second light generator. One or both of the light generators may be mountable on a section attachable to a canister containing the medicant to be dispensed. Also, the medicant dispenser may have a first sensor and a second sensor. In such an embodiment, the first sensor may be arranged to switch the counter to an operating condition on receipt of light emitted from the first light generator; the second light generator and the second sensor being positionable such that medicant dispensed from the dispenser interrupts the light beam emitted from the second light generator thereby provoking a signal from the second sensor; the counter being arranged to detect variations in the signal from the second sensor indicative of dispensation of the medicant.

In a further preferred embodiment, the medicant dispenser further comprises a filter positionable such that light from one or other or both of the first and second light generators passes through the filter before being received by one or other or both of the first or second sensors.

A reflector to reflect light from the second light generator onto the second sensor may be included. Also, one or both of the sensors may be positionable adjacent one another in a face of a housing for retaining the device and the valve. Alternatively, the second light generator may be positionable on a stop for the valve.

According to a second aspect of the present invention there is provided a device for detecting that medicant has been released from a medicant dispenser, the device comprising:
a light generator for emitting a beam of light;
a sensor for sensing light emitted from the light generator; and
a counter coupled to the sensor for receiving a signal therefrom and for counting dispensation of the medicant;
the light generator, the sensor and the counter being arranged to be switchable between a rest condition and an operating condition; the light generator and the sensor being positioned such that medicant being dispensed interrupts the light beam emitted from the light generator thereby provoking a further signal from the sensor; the counter being arranged to detect variations in the further signal from the sensor indicative of dispensation of the medicant.

According to a third aspect of the present invention there is provided a method of monitoring dispensation of a medicant from a medicant dispenser, the medicant dispenser having a valve with an actuator movable between a valve closed and a valve open position and through which medicant is selectively released; the method comprising:
emitting a beam of light from a light generator;
sensing using a sensor the beam of light emitted from the light generator; and
receiving in a counter coupled to the sensor a signal from the sensor;
detecting in the counter variations in a further signal from the sensor due to interruption of the light beam emitted from the light generator and received by the sensor by medicant dispensed, the variations being indicative of dispensation of the medicant for counting dispensation of the medicant.

Preferably, the method further comprises switching using a signal from the sensor the counter to an operating condition on receipt of light emitted from the light generator prior to the step of detecting.

In a further preferred embodiment, the method further comprises switching the light generator to an operating condition using a signal from the counter prior to the step of detecting.

The method preferably further comprises activating power to the light generator using a switch.

Preferably, the step of emitting light from a light generator comprises emitting light using a light emitting diode (LED).

In a preferred embodiment, the method further comprises locating in a housing the valve, and a device comprising the light generator, the sensor and the counter.

Preferably, the method further comprises mounting the sensor and/or the light generator on opposing faces of the housing. Alternatively, the light generator may be mountable on a section attachable to a canister containing the medicant to be dispensed which is insertable in use in the housing.

In a further preferred embodiment, the method further comprises mounting the sensor on a face of the housing opposing the light generator, and mounting a filter adjacent the sensor such that light from the light generator passes through the filter before being received by the sensor.

In a preferred embodiment, the step of emitting comprises emitting light from a first light generator and a second light generator. In such an embodiment, one or both of the light generators may be mountable on a section attachable to a canister containing the medicant to be dispensed. Also, the step of sensing may comprise sensing using a first sensor and a second sensor.

In a preferred embodiment, the method further comprises arranging the first sensor to switch the counter to an operating condition on receipt of light emitted from the first light generator; and positioning the second light generator and the second sensor such that medicant dispensed from the dispenser interrupts the light beam emitted from the second light generator thereby provoking a signal from the second sensor; and detecting using the counter variations in the signal from the second sensor indicative of dispensation of the medicant.

Preferably, the method further comprises positioning a filter such that light from one or other or both of the first and second light generators passes through the filter before being received by one or other or both of the first or second sensors and light from the second light generator may be reflected onto the second sensor using a reflector.

In a preferred embodiment, one or both of the sensors may be positioned adjacent one another in a face of a housing for retaining the dispenser and the valve. Also, the second light generator may be positioned on a stop for a valve of a medicant dispenser through which medicant is selectively released.

The present invention will now be described by way of example and with reference to the drawings in which:

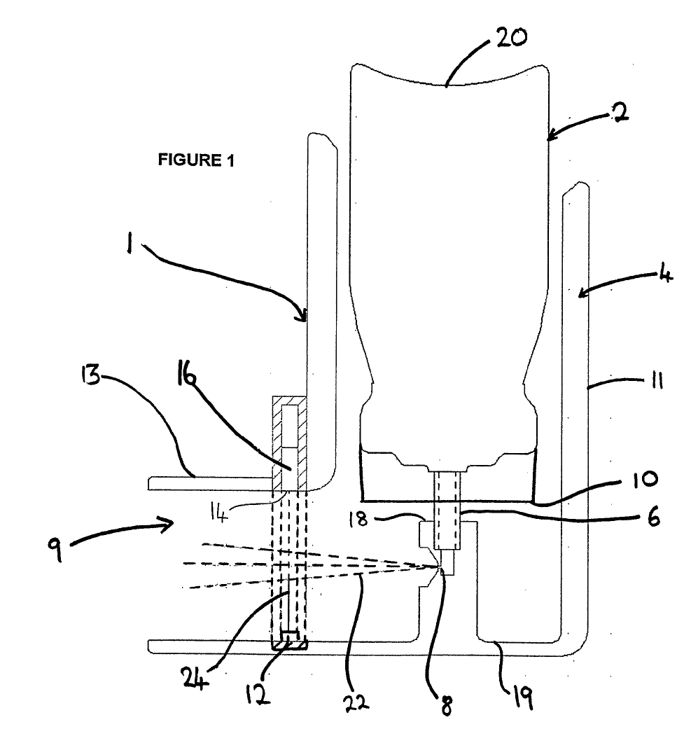

FIG. 1 shows in schematic cross-section a medicant dispenser in accordance with a first preferred embodiment of the present invention;

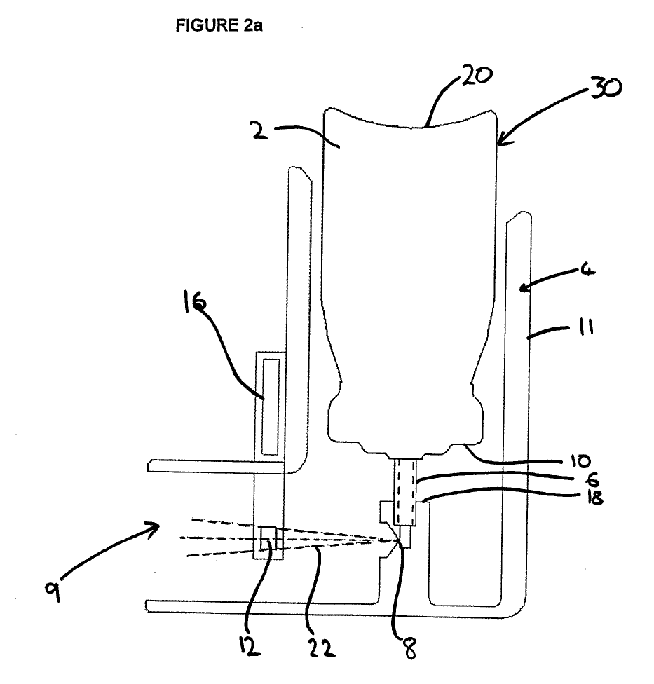

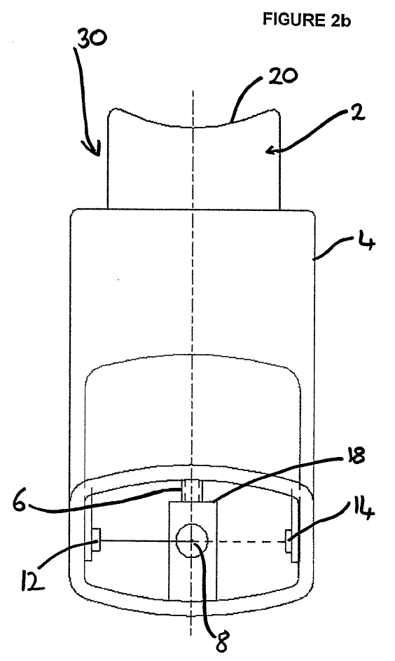

FIG. 2a shows in schematic cross-section a medicant dispenser in accordance with a second preferred embodiment of the present invention;

FIG. 2b shows in schematic a view into the outlet of the system of FIG. 2a;

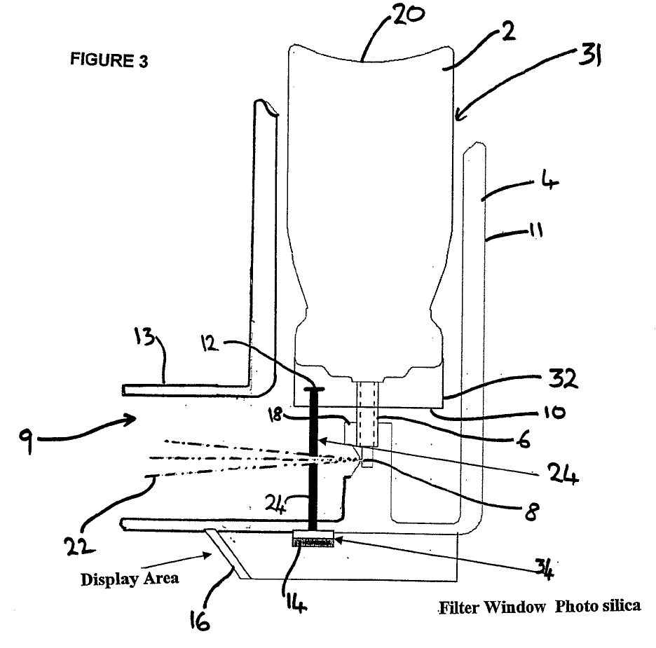

FIG. 3 shows in schematic cross-section a medicant dispenser in accordance with a third preferred embodiment of the present invention;

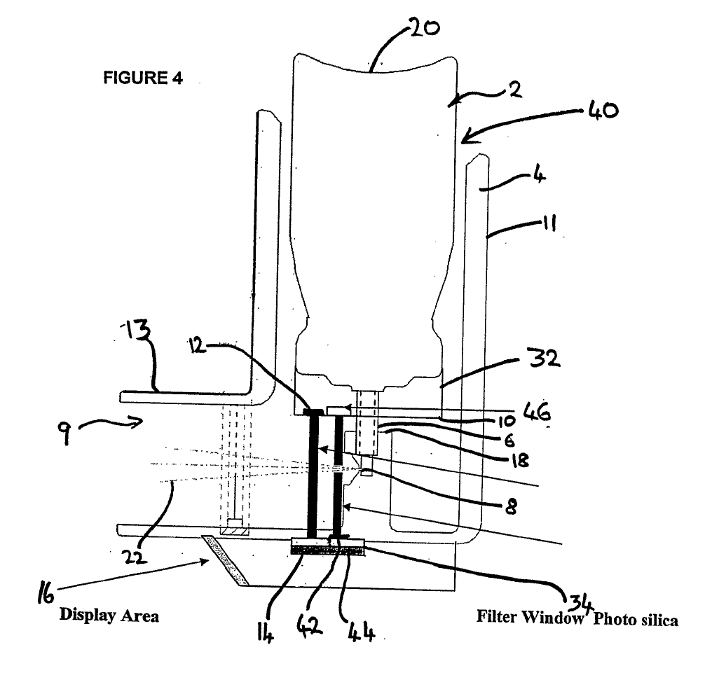

FIG. 4 shows in schematic cross-section a medicant dispenser in accordance with a fourth preferred embodiment of the present invention; and

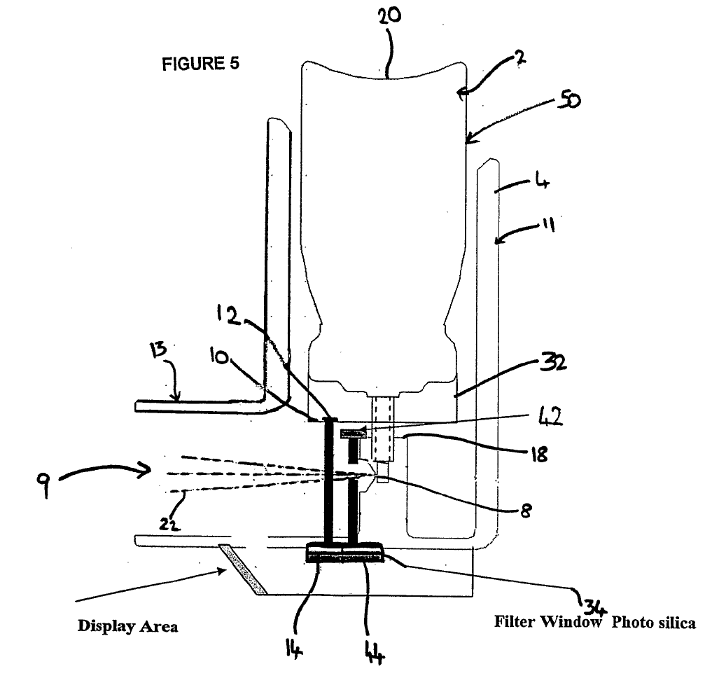

FIG. 5 shows in schematic cross-section a medicant dispenser in accordance with a fifth preferred embodiment of the present invention.

In a first preferred embodiment as shown in FIG. 1, the system 1 comprises a pressurised canister 2 mounted in a housing 4 together with a valve mechanism having a stem 6. The valve mechanism has an outlet 8 for permitting the dispensation of the medicant from the pressurised canister 2 and the system 1 has an outlet 9 through which the medicant is dispensed from the system 1. The outlet 9 of the system 1 may be inserted into the mouth of a user in use.

The valve mechanism is attached to a first end 10 of the pressurised canister 2 and this end 10 together with the valve mechanism is inserted into and retained within the housing. The housing 4 is preferably in the form of a substantially L-shaped hollow tube and the valve mechanism and canister 2 are preferably retained in one arm 11 of the housing 4. A light generator, such as an LED 12, is mounted on the inner base face of the second arm 13 of the housing 4 between the outlet 9 of the system and the valve mechanism. A sensor 14 is mounted on an opposing inner face of the housing 4 to the LED 12 and is arranged to receive a beam of light emitted from the LED 12. A counter 16 and its associated electronics and display may be mounted on an external face of the housing, for example at a location adjacent to the sensor 14.

In operation, the canister 2 together with the valve mechanism attached thereto is inserted into the housing 4 such that the end 10 of the canister 2 to which the valve mechanism is attached, together with the valve mechanism, are retained within the housing 4. The stem 6 of the valve mechanism rests at its free end against a stop 18 projecting from the inner base face 19 of the housing 4. In standby, the electronics of the counter 16 are in sleep mode.

To operate the system 1 to dispense medicant, the free end 20 of the canister 2 is pressed in a direction towards the base of the housing 4. As the canister 2 is depressed, and before the valve mechanism is fully opened, a switch (not shown) is operated which activates the LED 12. The beam emitted from the LED 12 is then detected by the sensor 14 and wakes the electronics associated with the counter 16. On further depression of the canister 2, the stem 6 of the valve mechanism is pushed by the stop 18 into the canister 2 thereby opening the valve. The medicant is forced under pressure out of the outlet 8 in the valve mechanism and is carried in spray form 22 towards the outlet 9 of the system 1. As the spray 22 passes through the beam 24, the signal received by the sensor 14 is reduced causing a signal to be sent to the counter 16 to record the fact that a dose has been dispensed. Should the contents of the canister 2 be below a predetermined level, no medicant will be emitted and therefore no medicant will disrupt the beam. In such a case, although the electronics will have been activated by the LED 12, no count will be recorded. After dispensing of the dose, the electronics reverts to sleep mode. As the canister returns to its normal position, the LED 12 is preferably automatically switched off.

Alternatively, in a further preferred embodiment, as the canister 2 is depressed, and before the valve mechanism is fully opened, a switch (not shown) is operated which activates the counter 16. The counter 16 then sends a signal to the light generator 12 to activate the light generator. A beam is then emitted from the light generator 12 and this is detected by the sensor 14. The operation is then as described above with regard to the first preferred embodiment.

A further preferred embodiment is shown in FIGS. 2a and 2b and the same reference numerals have been used as in FIG. 1 to denote identical components in the two systems. The system 30 of FIGS. 2a and 2b differs from that of FIG. 1 in that the LED 12 and the sensor 14 are mounted on opposing inner side faces of the first arm of the housing 4 rather than the LED and sensor being mounted on an inner base face of the second arm of the housing. Thus, in the embodiment of FIGS. 2a and 2b, the light beam emitted from the LED 12 projects in a plane substantially perpendicular to that in which the beam projects in the embodiment of FIG. 1.

The operation of the system 30 of FIGS. 2a and 2b is essentially the same as that described above with regard to FIG. 1.

In further preferred embodiments, a battery may be included in or coupled to the light generator 12 of the embodiments shown in FIGS. 1, 2a and 2b. The battery serves to power the light generator 12 on its activation by either the switch directly or the counter 16, depending on which signal is to be used for activation of the system. This is particularly advantageous as it enables more power to be available to detect the attenuation by the dispensed medicant of the light emitted from the light generator 16. Also, it enables the time period for detection of this attenuation to be greater than if the battery used to power the counter 16 is also used to power the light generator 12.

FIG. 3 shows a further preferred embodiment and, again, the same reference numerals have been used as in FIGS. 1, 2a and 2b to denote identical components in the systems. The system 31 of FIG. 3 differs from that of FIG. 1 in that the LED 12 is mounted on a section 32 of the canister 2 at a location on the first end 10 adjacent the valve mechanism. Furthermore, the sensor 14 in the system of FIG. 3 is mounted on an opposing outer face of the second arm of the housing 4 with a filter 34 being located between the sensor 14 and the housing 4 so that only light emitted from the LED will be detected by the sensor 14. The display 16 and its associated electronics are mounted on the outer base face of the second arm of the housing 4 adjacent the sensor 14.

In operation, the system 31 of FIG. 3 is essentially the same as that described above with regard to FIG. 1.

A further preferred embodiment is shown in FIG. 4. In this embodiment, the system 40 differs from that of FIG. 3 in that the system 40 comprises a second LED 42 mounted adjacent the first sensor 14 on an outer face of the second arm of the housing together with and adjacent to a second sensor 44. A reflector 46 is mounted on a section 32 of the canister 2 at a location on the first end 10 adjacent the valve mechanism such that the light beam from the second LED 42 is reflected back to the second sensor 44 by the reflector 46.

In FIG. 4, the same reference numerals as those used in the previous figures have been used to denote identical components in the systems.

In operation, the system 40 of FIG. 4 is similar to that of the system 31 of that shown in FIG. 3, in that depression of the canister 2 causes a switch (not shown) to be operated which activates the first LED 12, the beam from which is detected by the sensor 14 which then wakes the electronics associated with the counter 16 and turns on the second LED 42. On further depression of the canister 2, the stem 6 of the valve mechanism is pushed by the stop 18 into the canister 2 thereby opening the valve. The medicant is forced under pressure out of the outlet 8 in the valve mechanism and is carried in spray form 22 towards the outlet 9 of the system 40. As the spray 22 passes through the beam emitted from the second LED 42, the signal received by the second sensor 44 is reduced causing a signal to be sent to the counter 16 to record the fact that a dose has been dispensed. Should the contents of the canister 2 be below a predetermined level, no medicant will be emitted and therefore will not disrupt the beam and therefore although the electronics will be activated, no count will be recorded. After dispensing of the dose, the electronics reverts to sleep mode. As the canister returns to its normal position, the LED is switched off.

FIG. 5 shows a further preferred embodiment of the invention. In FIG. 5, the same reference numerals as those used in the previous figures have been used to denote identical components in the systems. The system 50 of FIG. 5 differs from that of FIG. 4 in that there is no reflector 46 and the second LED 42 is mounted on the valve stop 18 of the housing 2 rather than being adjacent the second sensor 44 as in the embodiment of FIG. 4. In operation, depression of the canister 2 causes a switch (not shown) to be operated which activates the first LED 12, the beam from which is detected by the first sensor 14 and which then wakes the electronics associated with the counter 16 and turns on the second LED 42. On further depression of the canister 2, the stem 6 of the valve mechanism is pushed by the stop 18 into the canister 2 thereby opening the valve. The medicant is forced under pressure out of the outlet 8 in the valve mechanism and is carried in spray form 22 towards the outlet 9 of the system 40. As in the system of FIG. 4, the spray 22 passes through the beam emitted from the second LED 42, the signal received by the second sensor 44 is reduced causing a signal to be sent to the counter 16 to record the fact that a dose has been dispensed. Should the contents of the canister 2 be below a predetermined level, no medicant will be emitted and therefore will not disrupt the beam and therefore although the electronics will be activated, no count will be recorded. After dispensing of the dose, the electronics reverts to sleep mode. As the canister returns to its normal position, the LED is switched off.

One or more preferred embodiments of the present invention provide an uncomplicated and economical system for dispensing medicant in which the dispensation of a dose of medicant is monitored.

Various modifications to the embodiments of the present invention described above may be made. For example, whilst a light emitting diode (LED) has been described above as a preferred light generator for use in preferred embodiments of the present invention, it will be appreciated that an alternative light generator may be used in the present invention. Also, the number of light generators and sensors used in a preferred embodiment may be varied, from that described above. Furthermore, whilst the term sensor has been used in embodiments of the invention and description thereof, it will be appreciated that any form of receiver for receiving a signal may be used. Other components and method steps may be added or substituted for those described above. Thus, although the invention has been described above using particular embodiments, many variations are possible within the scope of the claims, as will be clear to the skilled reader, without departing from the invention.

1. A medicant dispenser comprising:
a valve with an actuator movable between a valve closed and a valve open position and through which medicant is selectively released; and
a device for detecting that medicant has been released; wherein the device comprises:
a light generator for emitting a beam of light;
a sensor for sensing light emitted from the light generator; and
a counter coupled to the sensor for receiving a signal therefrom and for counting dispensation of the medicant;
the light generator, the sensor and the counter being arranged to be switchable between a rest condition and an operating condition; the light generator and the sensor being positioned such that medicant being dispensed interrupts the light beam emitted from the light generator thereby provoking a further signal from the sensor; the counter being arranged to detect variations in the further signal from the sensor indicative of dispensation of the medicant.

2. The medicant dispenser of claim 1 further comprising a switch for activating power to the light generator.

3. The medicant dispenser of claim 1, wherein the light generator comprises a light emitting diode (LED).

4. The medicant dispenser of claim 1, wherein the sensor is arranged to switch the counter to the operating condition on receipt of light emitted from the light generator.

5. The medicant dispenser of claim 1, wherein the counter is arranged to switch the light generator to the operating condition.

6. The medicant dispenser of claim 1, further comprising a battery arranged to power the light generator.

7. The medicant dispenser of claim 1 further comprising a housing for retaining the device and the valve.

8. The medicant dispenser of claim 7, wherein the sensor and/or the light generator are mountable on opposing faces of the housing.

9. The medicant dispenser of claim 7, wherein the light generator is mountable on a section attachable to a canister containing the medicant to be dispensed which is insertable in use in the housing.

10. The medicant dispenser of claim 9, wherein the sensor is mountable on a face of the housing opposing the light generator and a filter is mountable adjacent the sensor, the filter being positionable such that light from the light generator passes through the filter before being received by the sensor.

11. The medicant dispenser of claim 1 comprising a first light generator and a second light generator.

12. The medicant dispenser of claim 11 wherein one or both of the light generators is/are mountable on a section attachable to a canister containing the medicant to be dispensed.

13. The medicant dispenser according to claim 11, comprising a first sensor and a second sensor.

14. The medicant dispenser of claim 13, wherein the first sensor is arranged to switch the counter to an operating condition on receipt of light emitted from the first light generator; the second light generator and the second sensor being positionable such that medicant dispensed from the dispenser interrupts the light beam emitted from the second light generator thereby provoking a signal from the second sensor; the counter being arranged to detect variations in the signal from the second sensor indicative of dispensation of the medicant.

15. The medicant dispenser of claim 13, further comprising a filter positionable such that light from one or other or both of the first and second light generators passes through the filter before being received by one or other or both of the first or second sensors.

16. The medicant dispenser of claim 13, further comprising a reflector to reflect light from the second light generator onto the second sensor.

17. The medicant dispenser of claim 13, wherein one or both of the sensors is/are positionable adjacent one another in a face of a housing for retaining the device and the valve.

18. The medicant dispenser of claim 13, wherein the second light generator is positionable on a stop for the valve.

19. A device for detecting that medicant has been released from a medicant dispenser, the device comprising:
a light generator for emitting a beam of light;
a sensor for sensing light emitted from the light generator; and
a counter coupled to the sensor for receiving a signal therefrom and for counting dispensation of the medicant;
the light generator, the sensor and the counter being arranged to be switchable between a rest condition and an operating condition; the light generator and the sensor being positioned such that medicant being dispensed interrupts the light beam emitted from the light generator thereby provoking a further signal from the sensor; the counter being arranged to detect variations in the further signal from the sensor indicative of dispensation of the medicant.

20. The device of claim 19 further comprising a switch for activating power to the light generator;

21. The device of claim 19, wherein the light generator comprises a light emitting diode (LED).

22. The device of claim 19, wherein the sensor is arranged to switch the counter to the operating condition on receipt of light emitted from the light generator.

23. The device of claim 19, wherein the counter is arranged to switch the light generator to the operating condition.

24. The device of claim 19, further comprising a battery arranged to power the light generator.

25. The device of claim 19, further comprising a housing for retaining the device and a medicant dispenser having a valve with an actuator movable between a valve closed and a valve open position and through which medicant is selectively released.

26. The device of claim 25, wherein the sensor and/or the light generator are mountable on opposing faces of the housing.

27. The device of claim 25, wherein the light generator is mountable on a section attachable to a canister containing the medicant to be dispensed which is insertable in use in the housing.

28. The device of claim 27, wherein the sensor is mountable on a face of the housing opposing the light generator and a filter is mountable adjacent the sensor, the filter being positionable such that light from the light generator passes through the filter before being received by the sensor.

29. The device of claim 19 comprising a first light generator and a second light generator.

30. The device of claim 29 wherein one or both of the light generators is/are mountable on a section attachable to a canister containing the medicant to be dispensed.

31. The device according to claim 29, comprising a first sensor and a second sensor.

32. The device of claim 31, wherein the first sensor is arranged to switch the counter to an operating condition on receipt of light emitted from the first light generator; the second light generator and the second sensor being positionable such that medicant dispensed from the dispenser interrupts the light beam emitted from the second light generator thereby provoking a signal from the second sensor; the counter being arranged to detect variations in the signal from the second sensor indicative of dispensation of the medicant.

33. The device of claim 31, further comprising a filter positionable such that light from one or other or both of the first and second light generators passes through the filter before being received by one or other or both of the first or second sensors.

34. The device of claim 31, further comprising a reflector to reflect light from the second light generator onto the second sensor.

35. The device of claim 31, wherein one or both of the sensors is/are positionable adjacent one another in a face of a housing for retaining the device and the valve.

36. The device of claim 31, wherein the second light generator is positionable on a stop for a valve of a medicant dispenser through which medicant is selectively released.

37. A method of monitoring dispensation of a medicant from a medicant dispenser, the medicant dispenser having a valve with an actuator movable between a valve closed and a valve open position and through which medicant is selectively released; the method comprising:
emitting a beam of light from a light generator;
sensing using a sensor the beam of light emitted from the light generator; and
receiving in a counter coupled to the sensor a signal from the sensor;
detecting in the counter variations in a further signal from the sensor due to interruption of the light beam emitted from the light generator and received by the sensor by medicant dispensed, the variations being indicative of dispensation of the medicant for counting dispensation of the medicant.

38. The method of claim 37, further comprising activating power to the light generator using a switch.

39. The method of claim 37, wherein the step of emitting light from a light generator comprises emitting light using a light emitting diode (LED).

40. The method of claim 37, wherein the sensor is arranged to switch the counter to the operating condition on receipt of light emitted from the light generator.

41. The method of claim 37, wherein the counter is arranged to switch the light generator to the operating condition.

42. The method of claim 37, further comprising powering the light generator by a battery therein or connectable thereto.

43. The method of claim 37, further comprising locating in a housing the valve and a device comprising the light generator, the sensor and the counter.

44. The method of claim 43, comprising mounting the sensor and/or the light generator on opposing faces of the housing.

45. The method of claim 43, comprising mounting the light generator on a section attachable to a canister containing the medicant to be dispensed which is insertable in use in the housing.

46. The method of claim 45, comprising mounting the sensor on a face of the housing opposing the light generator, and mounting a filter adjacent the sensor such that light from the light generator passes through the filter before being received by the sensor.

47. The method of claim 37 wherein the step of emitting comprises emitting light from a first light generator and a second light generator.

48. The method of claim 47 comprising mounting one or both of the light generators on a section attachable to a canister containing the medicant to be dispensed.

49. The method according to claim 47, wherein the step of sensing comprises sensing using a first sensor and a second sensor.

50. The method of claim 49, comprising arranging the first sensor to switch the counter to an operating condition on receipt of light emitted from the first light generator; and positioning the second light generator and the second sensor such that medicant dispensed from the dispenser interrupts the light beam emitted from the second light generator thereby provoking a signal from the second sensor; and detecting using the counter variations in the signal from the second sensor indicative of dispensation of the medicant.

51. The method of claim 49, further comprises positioning a filter such that light from one or other or both of the first and second light generators passes through the filter before being received by one or other or both of the first or second sensors.

52. The method of claim 49, further comprising reflecting light from the second light generator onto the second sensor using a reflector.

53. The method of claim 49, comprising positioning one or both of the sensors adjacent one another in a face of a housing for retaining the dispenser and the valve.

54. The method of claim 49, comprising positioning the second light generator on a stop for a valve of a medicant dispenser through which medicant is selectively released.

55-57. (canceled)
